(19) United States
(12) Patent Application Publication
(10) Pub. No.: US 2023/0035575 A1
(43) Pub. Date: Feb. 2, 2023

(54) ANALYSIS DEVICE, ANALYSIS METHOD, AND RECORDING MEDIUM

(71) Applicant: Konica Minolta, Inc., Tokyo (JP)

(72) Inventors: Shinsuke KATSUHARA, Tokyo (JP); Hitoshi Futamura, Tokyo (JP); Ryohei Ito, Tokyo (JP); Hirotake Minami, Tokyo (JP); Nodoka Iida, Tokyo (JP); Amai Shimizu, Tokyo (JP)

(21) Appl. No.: 17/812,048

(22) Filed: Jul. 12, 2022

(30) Foreign Application Priority Data
Jul. 30, 2021 (JP) 2021-125349

(51) Int. Cl. G16H 50/20; G06N 5/02; G16H 40/63

(52) U.S. Cl. CPC G16H 50/20; G06N 5/022; G16H 40/63

(57) ABSTRACT

An analysis device includes a hardware processor, an acquirer, and an outputter, wherein the hardware processor acquires first medically-related information obtained through computer processing performed on medical information, the hardware processor generates structured data by structuring unstructured data acquired from user-created information on the basis of the medical information, the acquirer acquires second medically-related information from the structured data, the hardware processor compares the acquired first medically-related information and the second medically-related information acquired by the acquirer, and the outputter outputs next step information on the basis of a comparison result from the comparing.

### CROSS-REFERENCE TO RELATED APPLICATIONS

The entire disclosure of Japanese Patent Application No. 2021-125349 filed on Jul. 30, 2021 is incorporated herein by reference in its entirety.

### BACKGROUND

### Technological Field

The present invention relates to an analysis device, an analysis method, and a recording medium.

### Description of the Related Art

In recent years, along with the development of artificial intelligence (AI) technology, analysis by AI has been introduced also to the medical field, and attempts are being made to use AI to support the analysis and diagnosis of medical information such as image diagnosis that has been performed by physicians in the past.

For example, JP 5501491 discloses a diagnostic support device that detects a difference between first medically-related information based on user-created information and second medically-related information obtained through computer processing, and causes a display to display the difference between the two in a display format according to the combination of a lesion name included in the first medically-related information and a lesion name included in the second medically-related information.

In the clinical practice of medicine, it is desirable to perform examinations and diagnoses appropriately and rapidly, and to make diagnosis more efficient and optimized to reduce the burden on physicians.

The introduction of AI analysis is expected to contribute to such more efficient and optimized diagnosis.

### SUMMARY

However, the technology disclosed in JP 5501491 merely indicates that there is a difference between the first medically-related information and the second medically-related information. In other words, the next step of how to deal with the presence or absence of such a difference has not been fully investigated. Consequently, there is a problem in that even if AI analysis is introduced, more efficient and optimized diagnosis is not necessarily achieved.

For example, after a primary radiologist interprets a medical image, it is not determined whether the medical image should be passed on for interpretation by a secondary radiologist in a subsequent step.

For this reason, a secondary interpretation step occurs in all cases, and the burden on the secondary radiologist is not reduced.

The present invention has been devised in the light of such problems in the technology of the related art described above, and an objective of the present invention is to provide an analysis device, an analysis method, and a recording medium that can present the next step to a user according to an analysis result or diagnostic result with respect to medical information.

To achieve at least one of the abovementioned objects, according to an aspect of the present invention, an analysis device reflecting one aspect of the present invention includes:

an analyzer that acquires first medically-related information obtained through computer processing performed on medical information;

a generator that generates structured data by structuring unstructured data acquired from user-created information on the basis of the medical information;

an acquirer that acquires second medically-related information from the structured data;

a comparison processor that compares the first medically-related information acquired by the analyzer and the second medically-related information acquired by the acquirer; and

an outputter that outputs next step information on the basis of a comparison result from the comparing by the comparison processor.

According to another aspect, an analysis method includes:

analyzing medical information through computer processing to acquire first medically-related information; generating structured data by structuring unstructured data acquired from user-created information on the basis of the medical information;

acquiring second medically-related information from the structured data;

comparing the first medically-related information acquired by the analyzing and the acquired second medically-related information; and

outputting next step information on the basis of a comparison result from the comparing.

According to another aspect, a recording medium storing a program causes a computer to perform:
analyzing medical information through computer processing to acquire first medically-related information; generating structured data by structuring unstructured data acquired from user-created information on the basis of the medical information;

acquiring second medically-related information from the structured data;

comparing the first medically-related information acquired by the analyzing and the acquired second medically-related information; and

outputting next step information on the basis of a comparison result from the comparing by the comparison processor.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is an overall configuration diagram of a medical imaging system according to the present embodiment;

FIG. 2 is a key block diagram illustrating a functional configuration of an embodiment of an analysis device according to the present invention;

FIG. 3 is a table illustrating an example of a case of structuring natural language which is unstructured data;

FIG. 4 is a flowchart illustrating an analysis process in a first QA pattern;

FIG. 5 is an explanatory diagram schematically illustrating a flow of the analysis process illustrated in FIG. 4;

FIG. 6 is a flowchart illustrating comparison processing; and

FIG. 7 is a flowchart illustrating an analysis process in a second QA pattern.

### DETAILED DESCRIPTION OF THE EMBODIMENTS

Hereinafter, an embodiment of an analysis device, an analysis method, and a recording medium according to the present invention will be described. However, the scope of the invention is not limited to the illustrated examples.

### Configuration of Medical Imaging System

An analysis device according to the present embodiment performs analysis and the like of medical information, namely a medical image, in a medical imaging system, for example.

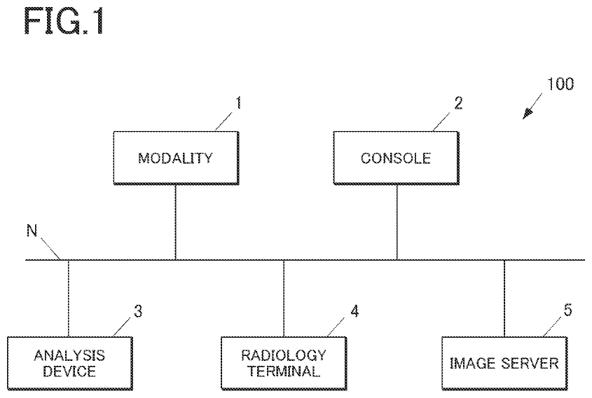

FIG. 1 illustrates a system configuration of the medical imaging system 100.

As illustrated in FIG. 1, the medical imaging system 100 includes a modality 1, a console 2, an analysis device 3, a radiology terminal 4, an image server 5, and the like, the above being connected over a communication network N such as a local area network (LAN), a wide area network (WAN), or the Internet. Each device included in the medical imaging system 100 conforms to the Health Level Seven (HL7) and Digital Image and Communications in Medicine (DICOM) standards, and communication between devices is performed in accordance with HL7 and DICOM. Note that the numbers of the modality 1, the console 2, the radiology terminal 4, and the like are not particularly limited.

The modality 1 is an image generation device such as an X-ray imaging device (DR, CR), an ultrasound diagnostic device (US), a CT, or an MRI, for example, and captures an image of a patient site to be examined as the subject and generates a medical image as medical information on the basis of examination order information transmitted from a radiology information system (RIS) or the like not illustrated. In the medical image generated in the modality 1, supplementary information (such as patient information, examination information, and an image ID) is written to, for example, the header of the image file in accordance with the DICOM standard. The medical image containing supplementary information in this way is transmitted to the analysis device 3 and the radiology terminal 4 through the console 2 or the like.

The console 2 is an imaging control device that controls imaging in the modality 1. The console 2 outputs imaging parameters and image scanning parameters to the modality 1, and acquires the image data of a medical image captured in the modality 1. The console 2 is provided with a controller, a display, an operating interface, a communication interface, a storage, and the like which are not illustrated, these components being connected by a bus.

The analysis device 3 is a device that performs any of various types of analysis on the medical image which is medical information. The analysis device 3 is configured as a PC or a mobile terminal, or as a dedicated device. In the present embodiment, a medical image management device such as a picture archiving and communication system (PACS), for example, is included in the analysis device 3.

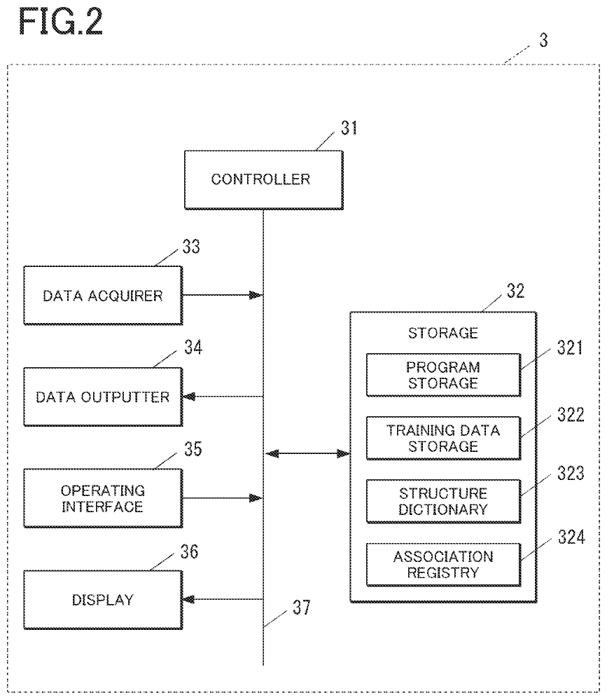

FIG. 2 is a block diagram illustrating a functional configuration of the analysis device 3.

As illustrated in FIG. 2, the analysis device 3 is provided with a controller 31 (hardware processor), a storage 32, a data acquirer 33, a data outputter 34, an operating interface 35, a display 36, and the like, these components being connected by a bus 37.

The data acquirer 33 is an acquirer that acquires various data from external devices (such as the console 2 and the radiology terminal 4 described later, for example).

The data acquirer 33 is configured as a network interface, for example, and is configured to receive data from external equipment connected in a wired or wireless manner through the communication network N. Note that in the present embodiment, the data acquirer 33 is configured as a network interface, but may also be configured as a port or the like into which USB memory, an SD card, or the like can be inserted.

In the present embodiment, the data acquirer 33 acquires the image data of a medical image from the console 2, for example. Additionally, the data acquirer 33 acquires information such as a diagnostic result (detection result information regarding a lesion that can be read from a medical image) related to a medical image created by a user (a physician, for example) on the basis of a medical image which is medical information from the radiology terminal 4 and a radiology report which is a radiology result by a radiologist (a radiologist who provides a primary interpretation or a secondary interpretation, for example). Also, if supplementary information is included, such as in the case where a region of interest (ROI) has been set in the medical image by a user such as a radiologist, the data acquirer 33 also acquires such supplementary information.

The data outputter 34 is an outputter that outputs information processed by the analysis device 3. The destination to which the data outputter 34 outputs various information is not particularly limited. For example, the destination may be the display 36 or the like of the analysis device 3, the radiology terminal 4 or the image server 5 described later, or any of various types of external display devices not illustrated.

For example, a network interface for communicating with the radiology terminal 4, the image server 5, or the like, a connector for connecting an external device (a display device or a printer not illustrated, for example), or a port for any of various media such as USB memory is applicable as the data outputter 34.

When a next step has been set on the basis of a comparison result from the controller 31 which functions as a comparison processor, for example, the data outputter 34 outputs information about the next step (also referred to as “next step information”).

The step information (next step information) output from the data outputter 34 may be information indicating whether or not to perform an additional examination and information indicating whether to treat the “first medically-related information” or the “second medically-related information” as “confirmed diagnosis” information, for example.

For example, in the case where the comparison result from the controller 31 functioning as a comparison processor indicates non-agreement between “first medically-related information” and “second medically-related information”, the data outputter 34 may output information requesting a diagnosis by a second user as the step information (next step information). As another example, in the case where the comparison result from the controller 31 functioning as a comparison processor indicates agreement between “first medically-related information” and “second medically-related information”, the data outputter 34 may output information not requesting a diagnosis by a second user as the step information (next step information). Here, the second user refers to a secondary radiologist (or final radiologist) who provides a secondary interpretation in the case where the “second medically-related information” is created by a primary radiologist who provides a primary interpretation.

As another example, request information asking a user (for example, the primary radiologist) to recheck the comparison result between the “first medically-related information” and the “second medically-related information” may also be output. With this arrangement, the user can grasp that a conclusion according to AI analysis is different from the user's own. Also, the handling in the case where the “first medically-related information” and the “second medically-related information” are not in agreement is not limited to requesting a diagnosis to a secondary radiologist (or final radiologist). For example, the user (for example, the primary radiologist) who created the “second medically-related information” may be requested to recheck the content of the diagnosis.

The operating interface 35 is configured as a keyboard provided with various keys, as a pointing device such as a mouse, as a touch panel attached to the display 36, or the like. Through the operating interface 35, the user is able to perform input operations. Specifically, an operation signal input via a key operation on the keyboard, a mouse operation, or a touch operation on the touch panel is output to the controller 31.

The display 36 is provided with a monitor such as a liquid crystal display (LCD), and displays various screens according to instructions in a display signal input from the controller 31. Note that the configuration is not limited to a single monitor, and a plurality of monitors may also be provided.

On the display 36, as described later, statistical information and the like output from the controller 31 (the controller 31 functioning as a comparison processor) is displayed as appropriate.

The controller 31 includes a central processing unit (CPU), random access memory (RAM), and the like, and centrally controls operations by the components of the analysis device 3. Specifically, the CPU reads out and loads various processing programs stored in a program storage 321 of the storage 32 into the RAM, and executes various processes according to the programs. In the present embodiment, the controller 31 achieves various functions as below through cooperation with the programs.

The controller 31 functions as an analyzer that acquires “first medically-related information” through computer processing performed on the medical information.

Specifically, lesion detection and analysis processing is performed on a medical image acquired by the data acquirer 33, and one or multiple lesion detection/analysis results are output as the “first medically-related information”. For the computer processing herein, artificial intelligence (AI) analysis using an AI that performs image diagnosis and image analysis, including lesion detection by computer-aided diagnosis (CAD), is used, for example.

The controller 31 also functions as a learner that learns correspondences between medical information (in the present embodiment, medical images) and medically-related information (such as names of lesions), for example, and the controller 31 functioning as the analyzer obtains the “first medically-related information” through computer processing performed on medical information (a medical image) on the basis of the correspondences between medical information (medical images) and medically-related information learned by the learner.

In other words, for example, a process of detecting/analyzing a lesion from an input medical image is performed by using a machine learning model created by using deep learning or the like to train the model with a large amount of training data (pairs of a medical image in which a lesion appears and a ground truth label (such as a lesion region in the medical images and a diagnostic name of the lesion (the type of lesion))).

The “first medically-related information” acquired in this way is information indicating the name, location, and the like of a lesion, for example, and is attached to the image data of the medical image as supplementary information.

Additionally, in the present embodiment, the controller 31 also functions as an acquirer (comparison target acquirer) that acquires a comparison target to be compared to the “first medically-related information” obtained by AI analysis.

The controller 31 functioning as the comparison target acquirer acquires the “second medically-related information” as a comparison target to be compared to the “first medically-related information” on the basis of information created by a user on the basis of medical information on the radiology terminal 4 or the like.

Note that the information created by the user (for example, a radiologist) is typically unstructured data.

Here, unstructured data refers to the medical information itself (such as a medical image or electrocardiogram waveform data, for example; in the present embodiment, a medical image in particular) or information (such as a radiology report, which is natural language created in relation to a medical image, for example) created by the user on the basis of the medical information, for example.

In contrast, the “first medically-related information” which is the result from AI analysis is structured data. For this reason, to compare the two, it is necessary to also structure the “second medically-related information” treated as the comparison target to obtain structured data containing character strings or the like that can be compared to the “first medically-related information”.

In the present embodiment, structuring includes analyzing text, an image, audio, or video, for example, and tagging metadata.

Also, in the present embodiment, structuring includes, for example, dividing unstructured data (for example, the natural language of radiology report information) acquired from user-created information (for example, radiology report information) into words (morphemes) and assigning meaning to the words on the basis of medical information (for example, a medical image). Assigning meaning to words includes classifying a sentence into a subject, verb, object, and complement (SVOC), for example. Assigning meaning to words also includes classifying words by attribute (location, finding, disease name, affirmative/negative, affirmative/negative determination, important finding, numerical value).

In the present embodiment, structured data refers to data that has been subjected to the above structuring.

In the present embodiment, the controller 31 functions as a generator that generates structured data by structuring unstructured data acquired from user-created information (for example, the natural language of a radiology report) on the basis of medical information (for example, a medical image).

Specifically, a structure dictionary 323 in which words are pre-classified into prescribed attributes is prepared in the storage 32, for example, and the controller 31 functioning as the generator classifies words included in the unstructured data by attribute and assigns meaning according to the structure dictionary 323. Note that the structure dictionary 323 may also be a dictionary obtained by machine learning.

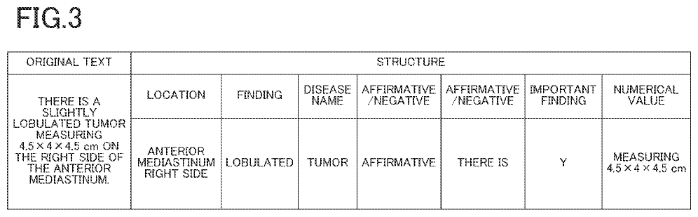

FIG. 3 illustrates an example of applying the structure dictionary 323 to divide and structure a radiology report (natural language which is unstructured data) into words.

As illustrated in FIG. 3, when the original text (natural language) of a radiology report states that “There is a slightly lobulated tumor measuring 4.5×4×4.5 cm on the right side of the anterior mediastinum.”, for example, the sentence indicating the location, size, and type of something is first divided into words. Accordingly, the location is the “right side” of the “mediastinum”, the size (numerical value) is “measuring 4.5×4×4.5 cm”, the finding is “lobulated”, and the disease name is “tumor”. The affirmative/negative is “affirmative” from the language “There is”, and corresponds to an important finding (“Y” in FIG. 3). By dividing the language into words assigned meaning in this way, the natural language which is unstructured data becomes structured data.

Additionally, the controller 31 also functions as an acquirer (comparison target acquirer) that acquires “second medically-related information” that can be compared to the “first medically-related information” from the structured data that has been structured in this way.

Moreover, the controller 31 functions as a comparison processor that compares the “first medically-related information” acquired by functioning as the analyzer and the structured “second medically-related information” acquired by functioning as the acquirer.

In other words, the two sets of information are checked against each other and a result of the comparison is output.

Specifically, it is clearly indicated whether the “first medically-related information” and the “second medically-related information” agree or do not agree (differ).

Since the “first medically-related information” and the “second medically-related information” are both structured data, comparison is possible by checking the two against each other.

The method by which the controller 31 functioning as the comparison processor compares the “first medically-related information” and the “second medically-related information” is not particularly limited.

As a premise for functioning as the comparison processor to compare the “first medically-related information” and the “second medically-related information”, the controller 31 may also function as a classifier that classifies the “second medically-related information” into information (herein referred to as “third medically-related information”) that is easily compared to the “first medically-related information”.

Here, the “second medically-related information” includes any of an image finding name, a disease name, or an anatomical location name. Image finding names, disease names, anatomical location names, and the like are expressed in a variety of different ways depending on the individual user (such as a radiologist). For this reason, it is effective to classify the “second medically-related information” into “third medically-related information” for checking against the “first medically-related information” to combine expressions into a single expression as much as possible.

For example, in the case where the user (such as a radiologist) uses expressions such as “nodular shadow”, “tumor”, and “light circular shadow”, the controller 31 functioning as the classifier combines all of these expressions into the expression “nodular shadow”. Thereafter, in the case where the “first medically-related information” which is the result from AI analysis contains the language “nodular shadow”, even if the original expression by the user (such as a radiologist) was “tumor” and the words themselves are not in agreement, the word “tumor” is also considered to have the same meaning as “nodular shadow”, and therefore the controller 31 functioning as the comparison process concludes that there is agreement with the analysis result from the AI.

Note that the controller 31 functioning as the classifier is not limited to classifying the “second medically-related information” into “third medically-related information”.

For example, when combining expressions included in the “second medically-related information” into one, the controller 31 functioning as the classifier may classify the “second medically-related information” into the “first medically-related information” which is the analysis result from the AI.

For example, in the case where the user (such as a radiologist) uses expressions such as “lung field, upper portion”, “upper lung field”, and “upper lung”, and the “first medically-related information” which is the analysis result from the AI consistently uses the expression “upper lung field”, the controller 31 functioning as the classifier combines all of the expressions into the expression “upper lung field” used in the analysis result from the AI.

With this arrangement, the “second medically-related information” and the “first medically-related information” can be compared with completely consistent terms, and the accuracy of checking the information against each other is improved.

Note that the method by which the controller 31 functioning as the classifier classifies the “second medically-related information” into the “third medically-related information” or classifies the “second medically-related information” into the “first medically-related information” is not particularly limited.

For example, the storage 32 is provided with an association registry 324 that registers correspondences between the “second medically-related information” and the “third medically-related information” or correspondences between the “second medically-related information” and the “first medically-related information”, and the controller 31 functioning as the classifier performs classification by referencing the association registry 324.

In addition, the controller 31 functioning as the classifier may also function as a learner that learns correspondences between the “second medically-related information” and the “first medically-related information”, and make associations between the two through learning.

In the case of making associations through learning, a similarity between the “second medically-related information” and the “first medically-related information” is calculated. In this case, the controller 31 also functions as a similarity calculator that calculates the similarity between the two.

For example, in the case where the “second medically-related information” or the like is structured text data, the similarity is obtained by performing text mining (similarity calculation) between the data of the “second medically-related information” and the “first medically-related information”.

For the similarity calculation, a method of creating a vector space model that uses vectors to express which words appear at what degree of frequency in a document can be used. With this method, by comparing vectors between the data to be compared with each other, the similarity between the data can be calculated. When calculating the similarity in this way, the controller 31 functioning as the similarity calculator preferably learns similarities between “second medically-related information” and “first medically-related information” in advance through machine learning or the like. Note that the method of calculating the similarity is not particularly limited, and any of various types of methods can be used.

Note that when the controller 31 analyzes medical information (for example, a medical image) as the analyzer, if words such as lesion information can be defined broadly to allow for flexible comparison, agreement or non-agreement can be determined with consideration for the definitions of the words included in the “first medically-related information”, even if the expressions used in the “first medically-related information” and the “second medically-related information” vary somewhat. In this case, it is not necessary to classify the “second medically-related information” into the “first medically-related information” or the “third medically-related information”, and comparison processing between the two is possible even if the controller 31 does not function as the classifier.

Note that in the case where the controller 31 functioning as the classifier can classify the “second medically-related information” by referencing information preregistered in the association registry 324, the classifier preferably classifies the “second medically-related information” on the basis of the information registered in the association registry 324, and functions as the learner to classify the “second medically-related information” through learning in the case where information is not registered in the association registry 324. With this arrangement, user intention can be prioritized in the case where the user has registered associations in advance.

Note that in the case where the structured “second medically-related information” acquired by the controller 31 functioning as the acquirer contains unknown lesion information, the information does not correspond to what is preregistered in the association registry 324, and the similarity to the “first medically-related information” which is the analysis result from the AI cannot be calculated.

In this case, the controller 31 functions as a lesion information classifier to classify the “second medically-related information” into the “first medically-related information”, and functions as a classification result presenter that presents the classification result from the lesion information classifier to the user for approval.

Specifically, for example, the words or the like included in the “second medically-related information” are analyzed to extract the words or the like determined to be closest to the words used in the analysis result from the AI registered in the association registry 324. Thereafter, the controller 31 outputs the classification result from the lesion information classifier to the display 36 or the like to present the classification result to the user as the classification result presenter, and asks the user to approve the classification result by indicating whether the unknown lesion information included in the “second medically-related information” may be associated with the extracted words.

As a result, if the user approves the classification result, the controller 31 functioning as the learner learns the unknown lesion information as prescribed lesion information in accordance with the classification result, and registers the learned result in the association registry 324.

With this arrangement, when the same lesion information is next read, the lesion information can be classified correctly by referencing the association registry 324.

The storage 32 is configured as a hard disk drive (HDD), semiconductor memory, or the like, and stores programs for executing various processes like the process for analyzing medical information such as medical images described later, and parameters, files, and the like necessary for the execution of the programs.

For example, besides the program storage 321 that stores various programs, a training data storage 322, the structure dictionary 323, the association registry 324, and the like are stored in the storage 32 of the present embodiment.

The training data storage 322 stores a large amount of training data and a machine learning model created by using deep learning or the like to train the model using the training data as described above, for example.

In addition, the structure dictionary 323 is dictionary data used to generate the “second medically-related information” which is structured data obtained by structuring a radiology report or the like created by the user (radiologist) as described above.

The association registry 324 is a correspondence table or the like in which correspondence relationships between words obtained when checking structured data against each other are registered in advance.

The radiology terminal 4 is a computer device provided with a controller, an operating interface, a display, a storage, and a communication interface, for example, and reads out medical information, namely a medical image, from the image server 5 or the like and displays the image for interpretation.

The user (primary radiologist, secondary radiologist) interprets the medical image on the radiology terminal 4 and creates a radiology report or the like which is a diagnostic result from the radiologist in relation to the medical image.

Moreover, the user (primary radiologist, secondary radiologist) may attach information indicating a region of interest (ROI), for example, to the medical image on the radiology terminal 4.

The information indicating a region of interest (ROI) is a mark, frame, or the like applied to a portion determined to be the site of a lesion, for example, and is set in the image by having the user (primary radiologist, secondary radiologist) designate a region by touching the display screen or using an operating interface such as a pointing device. The information indicating a region of interest (ROI) is information containing coordinate information or the like indicating a location or region, and is itself structured data generated on the basis of the medical information. In this case, the generator that generates structured data is a controller of the radiology terminal 4 that sets the information (coordinate information or the like) indicating the region of interest (ROI) according to an operation by the user (primary radiologist, secondary radiologist).

The image server 5 is a server forming a picture archiving and communication system (PACS), for example, and stores patient information (such as a patient ID, patient name, date of birth, age, sex, height, and weight) and examination information (such as an examination ID, examination date and time, type of modality, examination site, requested department, and purpose of examination) for a medical image output from the modality 1, an image ID of the medical image, information about the analysis result from the AI (that is, the “first medically-related information”) output from the controller 31 of the analysis device 3 and the “second medically-related information” which is the diagnostic result from the radiologist, such as a radiology report or the like created by the user (radiologist) on the radiology terminal 4, the result (comparison result) of checking the information against each other in the controller 31 (comparison processor) of the analysis device 3, and the like in association with each other in a database.

Also, the image server 5 reads out a medical image requested by the radiology terminal 4 and the various supplementary information attached to the medical image from the database, and causes the image and information to be displayed on the radiology terminal 4.

### Analysis Method in Present Embodiment

In the present embodiment, the analysis method includes: analyzing medical information such as a medical image through computer processing to acquire the “first medically-related information”; generating structured data by structuring unstructured data acquired from user-created information on the basis of the medical information; acquiring the “second medically-related information” from the generated structured data; comparing the “first medically-related information” acquired by the analyzing and the acquired “second medically-related information”; and outputting next step information on the basis of a comparison result from the comparing.

According to the analysis method in the present embodiment, by jointly considering the analysis result from the AI and the diagnostic result from the radiologist, quality assurance (hereinafter referred to as “QA”) can be performed for the diagnostic accuracy in relation to medical information (a medical image).

With regard to QA patterns, the flow of the analysis method is different depending on the degree to which the analysis result from the AI can be trusted.

A first QA pattern is a flow adopted in the case where the analysis result from the AI is not very reliable, and in the case where AI analysis cannot be trusted completely.

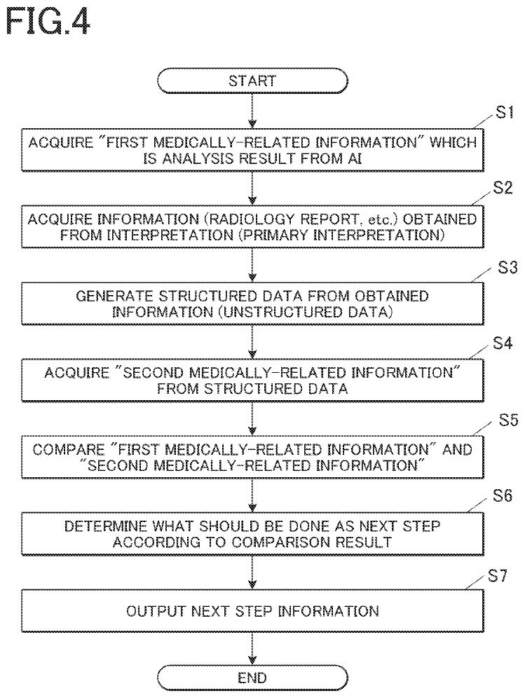

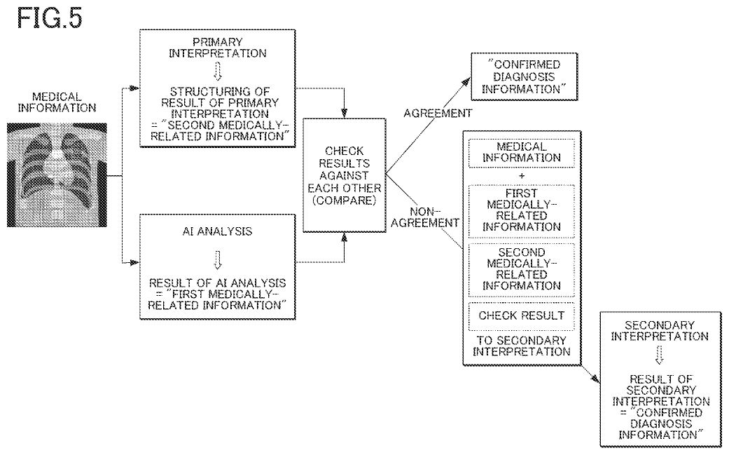

FIG. 4 is a flowchart illustrating an analysis process in the first QA pattern, and FIG. 5 is an explanatory diagram schematically illustrating the flow of processes.

As illustrated in FIGS. 4 and 5, in the first QA pattern, first, the medical information (medical image) to be processed is analyzed by an AI (AI application) in the controller 31 of the analysis device 3, and “first medically-related information” which is the analysis result (diagnostic result) is acquired (step S1; analyzing).

Also, information such as a radiology report created by having a user (a radiologist who provides a primary interpretation) interpret the medical information (here, a medical image) to be processed is acquired by the data acquirer 33 (step S2). Thereafter, the unstructured data acquired from the information is structured in the controller 31 functioning as the generator, structured data is generated (step S3; generating), and the controller 31 functioning as the acquirer acquires the “second medically-related information” from the structured data (step S4; acquiring).

Thereafter, the “first medically-related information” and the “second medically-related information” are checked against each other (compared) in the controller 31 functioning as the comparison processor (step S5; comparing).

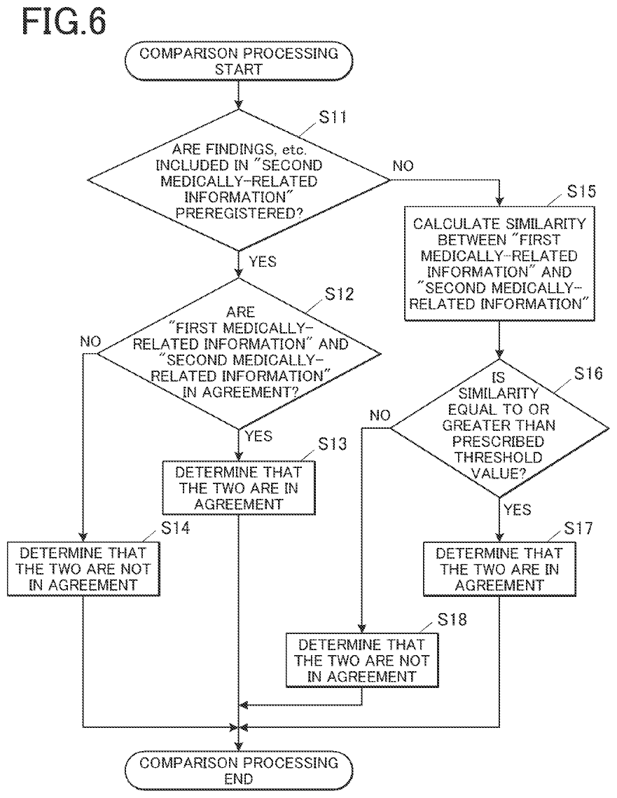

FIG. 6 is a flowchart illustrating comparison processing.

As illustrated in FIG. 6, in the comparison processing, first, the controller 31 determines whether lesion information such as a finding or a lesion name included in the “second medically-related information” is preregistered in the association registry 324 (step S11).

If the lesion information included in the “second medically-related information” is preregistered in the association registry 324 (step S11; YES), the controller 31 checks (compares) the “first medically-related information” and the “second medically-related information” against each other on the basis of the preregistered information, and determines whether the two are in agreement or non-agreement (that is, differ) (step S12). If the two are in agreement (step S12; YES), the “first medically-related information” and the “second medically-related information” are determined to be in agreement (step S13), whereas if the two are not in agreement (step S12: NO), the “first medically-related information” and the “second medically-related information” are determined to be in non-agreement (step S14).

On the other hand, if the lesion information included in the “second medically-related information” is not preregistered in the association registry 324 (step S11; NO), the controller 31 calculates the similarity between the “first medically-related information” and the “second medically-related information” through machine learning (step S15). Next, it is determined whether the calculated similarity is equal to or greater than a prescribed threshold value (step S16), and if the calculated similarity is equal to or greater than the prescribed threshold value (step S16; YES), the “first medically-related information” and the “second medically-related information” are determined to be in agreement (step S17). Also, if the calculated similarity is not equal to or greater than the prescribed threshold value (step S16; NO), the “first medically-related information” and the “second medically-related information” are determined to be in non-agreement (step S18).

Note that if the lesion information such as a finding or a lesion name included in the “second medically-related information” is unknown, the controller 31 classifies the lesion information into some words that appear in the analysis result from the AI, and presents the classification result to the user (for example, a radiologist) for approval. If approved, the classification result is registered in the association registry 324 as new association information. If a new association is registered in the association registry 324, the association registered in the association registry 324 is referenced the next time the same lesion information is input.

On the other hand, if not approved, the lesion information is repeatedly classified again into different words until the classification result is approved by the user.

Returning to FIGS. 4 and 5, if the “first medically-related information” and the “second medically-related information” are determined to be in agreement or non-agreement in the comparison processing, the controller 31 determines what should be done as the next step on the basis of the comparison result obtained by the comparing in the comparison processing (step S6), and the determination result is output from the data outputter 34 or the like as next step information (step S7; outputting).

For example, if the “first medically-related information” and the “second medically-related information” are in agreement, the information in agreement is treated as “confirmed diagnosis information”.

Also, if the two are in non-agreement, the passing of information to a second user (that is, a secondary radiologist or final radiologist different from the primary radiologist) is output as the “next step information”.

In this case, information such as the data of the medical image, the “first medically-related information”, the “second medically-related information”, and the check result (such as a determination of what kind of content is in agreement and what is in non-agreement, for example) is sent to the radiology terminal 4 operated by the secondary radiologist. Thereafter, the secondary radiologist refers to this information to make a secondary interpretation, and a diagnostic result such as a radiology report is created as the “second medically-related information (according to the secondary interpretation)”, which is treated as the “confirmed diagnosis information”.

Note that the “next step information” is not limited to the above, and if further examination is determined to be necessary (such as in the case where the “second medically-related information” based on a diagnostic result from the primary radiologist contains language indicating the need for reexamination, for example), content indicating additional examination or the like is output as the “next step information”.

In this way, in the process illustrated in FIGS. 4 and 5, the “second medically-related information” based on the diagnostic result from the primary radiologist and the “first medically-related information” which is the analysis result (diagnostic result) from the AI are checked against each other (compared), and the user can be informed of next step information according to the result (check result).

With this arrangement, in the case where the “first medically-related information” and the “second medically-related information” are in agreement, for example, the information is not passed on for a secondary interpretation, and the burden on the secondary radiologist is reduced. Consequently, the secondary radiologist is able to concentrate on cases that need a secondary interpretation, and can provide efficient and appropriate interpretations.

Moreover, since unstructured data such as a user-created radiology report is structured and then compared to the “first medically-related information” which is the analysis result from the AI, appropriate comparison processing can be performed.

When “confirmed diagnosis information” is obtained for the medical image treated as the medical information, the medical image is stored in the image server 5 in association with the “confirmed diagnosis information” confirmed in the analysis device 3.

Note that the save locations for the medical image, the various information attached thereto (such as the “first medically-related information” and the “second medically-related information”), the “confirmed diagnosis information”, and the like are not particularly limited.

For example, in the case where the analysis device 3, the radiology terminal 4, and the image server 5 form a PACS, the above information may be saved and managed collectively in an information management server or the like on the PACS.

Moreover, not all of the above information has to be saved, and for example, only the medical image and the “confirmed diagnosis information” may be saved as a set.

Note that the “confirmed diagnosis information” referred to herein is a diagnostic result obtained by having a physician make a definite conclusion from the medical image and the findings and analysis result obtained on the basis thereof.

In some cases, the final diagnosis of the patient is made by, for example, a clinician comprehensively considering the conclusion reached by the physician who created the “confirmed diagnosis information” in addition to examination data, observation data, and the like obtained through various examinations, observations, and so on.

Note that, unlike the above, in cases where the reliability of the analysis result from the AI is remarkably high or the AI analysis can be trusted completely, the flow of a second QA pattern is adopted.

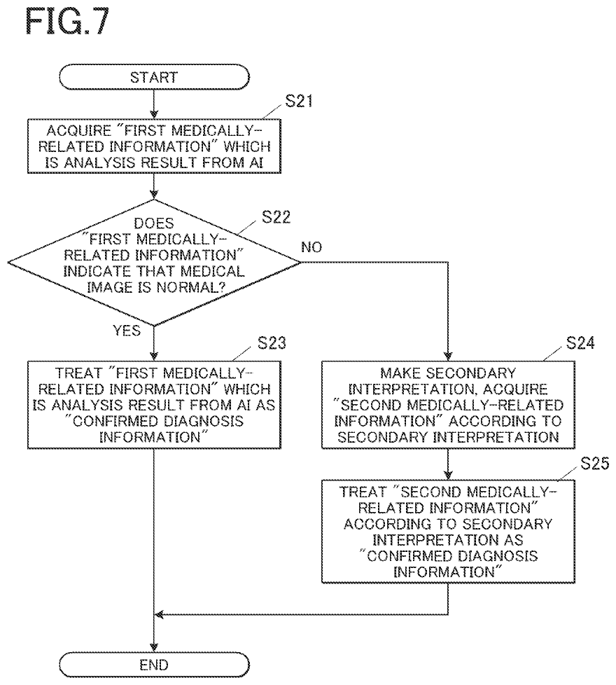

FIG. 7 is a flowchart illustrating an analysis process in the second QA pattern.

As illustrated in FIG. 7, in the second QA pattern, first, the medical information (medical image) to be processed is analyzed by an AI in the controller 31 of the analysis device 3, and “first medically-related information” which is the analysis result (diagnostic result) is acquired (step S21).

Note that in this case, the acquisition of “second medically-related information (according to the primary interpretation)” which is the diagnostic result from the primary radiologist is not essential.

Thereafter, the controller 31 determines whether the “first medically-related information” indicates that the medical image is normal (step S22). In other words, it is determined whether the “first medically-related information” includes some kind of abnormal finding with regard to the medical image.

If the “first medically-related information” indicates that the medical image is normal (step S22; YES), the “first medically-related information” which is the diagnostic result from the AI is treated as “confirmed diagnosis information” (step S23).

In this case, the radiologist can be saved from the work of interpreting medical images that the AI has concluded to be normal, the burden on the radiologist can be reduced, and efficient image diagnosis can be performed.

Note that even if the “first medically-related information” indicates that the medical image is normal, this result may not be treated as the “confirmed diagnosis information” immediately, and instead, a “normal label” indicating that the medical image is normal and does not include an abnormal finding may be attached to the “first medically-related information” which is the analysis result from the AI or the like, the medical image to be determined and the “first medically-related information” may be passed to a secondary radiologist (or a final radiologist), and a diagnostic result from the secondary radiologist may be treated as the “confirmed diagnosis information”.

In this case, a diagnosis by the secondary radiologist (final radiologist) is still respected, but by attaching the “normal label”, the burden on the radiologist can be reduced.

Note that the situations where the second QA pattern is applied are not limited to cases where the analysis result from the AI can be trusted completely, and may also include cases where the scope of trust is limited.

In other words, for example, if it is necessary to interpret a single image from different viewpoints, the diagnosis for a specific lesion may be entrusted to AI analysis and the “first medically-related information” which is the analysis result may be trusted, whereas for all other lesions, the “first medically-related information” which is the analysis result from the AI and the “second medically-related information” based on a diagnostic result from the radiologist may be used jointly.

Specifically, in the case where an X-ray image from a health screening or the like is extended to capture the thoracoabdominal area (or the long upper body), the “first medically-related information” which is the analysis result from the AI may be trusted with regard to the diagnosis of the thoracic area, but the “first medically-related information” which is the analysis result from the AI and the “second medically-related information” based on a diagnostic result from the radiologist may be used jointly for the abdominal area.

In particular, the “medical image” is not limited to a still image and may also be a moving image in some cases. In such cases, the number of diagnostic elements is increased over a still image. For this reason, even if limited AI analysis is applied in different ways depending on the clinical department and the target of diagnosis, such as trusting the “first medically-related information” which is the analysis result from the AI as a general rule in the diagnosis of the respiratory system and jointly using the “first medically-related information” which is the analysis result from the AI and the “second medically-related information” based on a diagnostic result from the radiologist in the diagnosis of the circulatory (blood flow) system, for example, an effect of increasing the efficiency of diagnosis, reducing the burden on the radiologist, and the like can be expected.

On the other hand, in the case where the “first medically-related information” does not indicate that the medical image is normal (the case where an abnormal finding is included, step S22; NO), the medical image to be determined and the “first medically-related information” are passed to the secondary radiologist (or final radiologist) who provides a secondary interpretation (or final interpretation) (step S24). Thereafter, the diagnostic result from the secondary radiologist is treated as the “confirmed diagnosis information” (step S25).

In this case, too, the controller 31 functioning as the comparison processor may check the “first medically-related information” which is the analysis result from the AI and the “confirmed diagnosis information” which is the diagnostic result from the secondary radiologist against each other to determine agreement or non-agreement. When the check result is output, the check result may also be stored in the image server 5 or the like together with the medical image and the “confirmed diagnosis information”. By storing the check result in association with the medical image and the “confirmed diagnosis information”, the information can be referred to later when a final diagnosis is made by the clinician or the like.

### Effects

As described above, the analysis device 3 according to the present embodiment includes: the controller 31 as an analyzer that acquires the “first medically-related information” obtained through computer processing (that is, AI analysis) performed on medical information; the controller 31 as a generator that generates structured data by structuring unstructured data acquired from user-created information on the basis of the medical information; the controller 31 as an acquirer that acquires the “second medically-related information” from the structured data; the controller 31 as a comparison processor that compares the “first medically-related information” and the “second medically-related information” acquired by the controller 31; and an outputter (data outputter 34) that outputs next step information on the basis of the comparison result.

By comparing the diagnosis from the radiologist to the analysis result from the AI and indicating the next step to the user according to the comparison result, efficient and effective diagnosis can be performed. In the case of image diagnosis in particular, it is determined whether or not a secondary interpretation is to be performed depending on the comparison result with the analysis result from the AI. For this reason, the burden on the secondary radiologist can be reduced, and efficient image diagnosis can be performed.

If the step information output from the outputter (data outputter 34) is information indicating whether or not an additional examination is to be performed, a necessary examination can be presented to the user according to the result of checking the “first medically-related information” and the “second medically-related information” against each other.

Consequently, the user can grasp the necessary step appropriately.

In addition, the step information output from the outputter (data outputter 34) can present, to the user, an indication regarding whether the “first medically-related information” which is the analysis result from the AI or the “second medically-related information” which is information based on the result of the interpretation by the radiologist is to be treated as a confirmed diagnosis, or whether the information should be passed to a secondary radiologist.

Consequently, the user can recognize cases where the “first medically-related information” or the like is treated as a confirmed diagnosis, and avoid the trouble involved in passing all image diagnoses to the secondary radiologist.

Additionally, in the case where comparison result of the comparing by the controller 31 functioning as the comparison processor indicates that the “first medically-related information” and the “second medically-related information” are not in agreement, information requesting a diagnosis by a second user (for example, a secondary radiologist) is output as the step information.

Consequently, the user can recognize cases where passing the information on for a secondary interpretation is necessary, and avoid the trouble involved in passing all image diagnoses to the secondary radiologist.

Also, the controller 31 may function as a classifier that classifies the “second medically-related information” into “third medically-related information”, and the “first medically-related information” and the “third medically-related information” may be compared.

The content and expressions used in a radiology report or the like vary considerably depending on the user (radiologist), and do not necessarily correspond to the results of AI analysis in some cases. Even in such cases, input variations among individual users can be made consistent, and accurate checking (comparing) with the analysis result from the AI can be achieved.

Also, a registry (association registry 324) that preregisters correspondences between the “second medically-related information” and the “third medically-related information” is provided, and in the case where the controller 31 functioning as the classifier classifies the “second medically-related information” into the “third medically-related information” on the basis of the correspondences between the “second medically-related information” and the “third medically-related information” registered in the association registry 324, classification that reflects preregistered user intentions can be performed.

Also, correspondences between the “second medically-related information” and the “third medically-related information” are learned, and in the case where the controller 31 functioning as the classifier classifies the “second medically-related information” into the “third medically-related information” on the basis of the learned correspondences between the “second medically-related information” and the “third medically-related information”, classification can be performed appropriately even if there are no preregistered correspondences.

Also, the controller 31 may function as a classifier that classifies the “second medically-related information” into the “first medically-related information”, and the “first medically-related information” and the classified “first medically-related information” may be compared.

The content and expressions used in a radiology report or the like vary considerably depending on the user (radiologist), and do not necessarily correspond to the results of AI analysis in some cases. Even these cases, input variations among individual users can be made consistent, and accurate checking (comparing) with the analysis result from the AI can be achieved.

Also, a registry (association registry 324) that preregisters correspondences between the “second medically-related information” and the “first medically-related information” is provided, and in the case where the controller 31 functioning as the classifier classifies the “second medically-related information” into the “first medically-related information” on the basis of the correspondences between the “second medically-related information” and the “first medically-related information” registered in the association registry 324, classification that reflects preregistered user intentions can be performed.

Also, correspondences between the “second medically-related information” and the “first medically-related information” are learned, and in the case where the controller 31 functioning as the classifier classifies the “second medically-related information” into the “first medically-related information” on the basis of the learned correspondences between the “second medically-related information” and the “first medically-related information”, classification can be performed appropriately even if there are no preregistered correspondences.

Also, in the case where there are no preregistered correspondences in the association registry 324, if the controller 31 functioning as the classifier classifies the “second medically-related information” into the “first medically-related information” on the basis of learned correspondences between the “second medically-related information” and the “first medically-related information”, classification can be performed appropriately while also reflecting user intentions, even if there are no preregistered correspondences.

In addition, a similarity calculator that calculates a similarity between the “second medically-related information” and the “first medically-related information” may also be provided, and in this case, correspondences between the “first medically-related information” and the “second medically-related information” are learned on the basis of the similarity.

With this arrangement, correspondences between the “second medically-related information” and the “first medically-related information” can be learned appropriately.

Also, in the case where the “second medically-related information” is unknown lesion information, the controller 31 may function as a lesion information classifier to classify the “second medically-related information” into the “first medically-related information” and present the classification result to the user for approval. In this case, if the classification result is approved, the unknown lesion information is learned as prescribed lesion information according to the classification result and registered in the registry (association registry 324).

With this arrangement, classification using the information registered in the association registry 324 can be performed the next time, and efficient and appropriate classification can be performed.

Also, in the case where position information or the like is automatically attached as supplementary information on the radiology terminal 4 or the like, such as when the user (a radiologist or the like) assigns a region of interest (ROI) to a medical image, the medical image is acquired by the analysis device 3 in a form with structured data attached.

According to the present embodiment, the data attached in a structured manner in this way is also included in the “second medically-related information” to be compared to the “first medically-related information”, and can be treated as a target of comparison appropriately.

### Modifications

Note that although an embodiment of the present invention has been described above, the present invention is not limited to such an embodiment, and obviously various modifications are possible within a scope that does not depart from the gist of the present invention.

For example, the above embodiment illustrates an example of a case where the medical information to be analyzed by the analysis device 3 is a medical image, but the medical information is not limited to a medical “image”.

Information or the like acquired by various examinations of a patient may be broadly included in the medical information, and for example, results obtained from any of various types of examinations, such as electrocardiogram waveform data and cardiac sound data, data related to blood flow, and the like may be included in the medical information.

Also, in the above embodiment, the analysis device 3, the radiology terminal 4, and the image server 5 are illustrated as respectively separate devices in FIG. 1, but the analysis device 3 and the image server 5, or the analysis device 3, the radiology terminal 4, and the image server 5, may also be configured as a single device or a single system.

Note that the present invention obviously is not limited to the above embodiment, modifications, and the like, and alterations can be made, as appropriate, without departing from the gist of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. An analysis device comprising:
a hardware processor;
an acquirer; and
an outputter, wherein
the hardware processor acquires first medically-related information obtained through computer processing performed on medical information,
the hardware processor generates structured data by structuring unstructured data acquired from user-created information on the basis of the medical information,
the acquirer acquires second medically-related information from the structured data,
the hardware processor compares the acquired first medically-related information and the second medically-related information acquired by the acquirer, and
the outputter outputs next step information on the basis of a comparison result from the comparing.

2. The analysis device according to claim 1, wherein the outputter outputs, as the step information, information indicating whether an additional examination is to be performed.

3. The analysis device according to claim 1, wherein the outputter outputs, as the step information, information indicating whether the first medically-related information or the second medically-related information is to be treated as a confirmed diagnosis.

4. The analysis device according to claim 1, wherein if the comparison result from the comparing indicates that the first medically-related information and the second medically-related information are not in agreement, the outputter outputs information requesting a diagnosis by a second user as the step information.

5. The analysis device according to claim 1, wherein the hardware processor
classifies the second medically-related information acquired by the acquirer into third medically-related information, and
compares the first medically-related information acquired by the acquirer and the third medically-related information classified by the classifier.

6. The analysis device according to claim 5, further comprising:
a storage in which correspondences between the second medically-related information and the third medically-related information are preregistered, wherein
the hardware processor classifies the second medically-related information acquired by the acquirer into the third medically-related information on the basis of the correspondences between the second medically-related information and the third medically-related information registered in the storage.

7. The analysis device according to claim 5, wherein
the hardware processor learns correspondences between the second medically-related information and the third medically-related information, and
the hardware processor classifies the second medically-related information acquired by the acquirer into the third medically-related information on the basis of the learned correspondences between the second medically-related information and the third medically-related information.

8. The analysis device according to claim 1, wherein
the hardware processor classifies the second medically-related information acquired by the acquirer into the first medically-related information, and
the hardware processor compares the first medically-related information and the classified first medically-related information.

9. The analysis device according to claim 8, further comprising:
a storage in which correspondences between the first medically-related information and the second medically-related information are preregistered, wherein
the hardware processor classifies the second medically-related information acquired by the acquirer into the first medically-related information on the basis of the correspondences between the first medically-related information and the second medically-related information registered by the registry.

10. The analysis device according to claim 8, wherein
the hardware processor learns correspondences between the first medically-related information and the second medically-related information, and
the hardware processor classifies the second medically-related information acquired by the acquirer into the first medically-related information on the basis of the learned correspondences between the first medically-related information and the second medically-related information.

11. The analysis device according to claim 9, wherein
the hardware processor learns correspondences between the first medically-related information and the second medically-related information, and
if correspondences between the first medically-related information and the second medically-related information are not registered in the storage,
the classifier classifies the second medically-related information acquired by the acquirer into the first medically-related information on the basis of the learned correspondences between the first medically-related information and the second medically-related information.

12. The analysis device according to claim 11, wherein
the hardware processor is pretrained to calculate a similarity between the second medically-related information acquired by the acquirer and the first medically-related information, and
the hardware processor learns correspondences between the first medically-related information and the second medically-related information based on the similarity.

13. The analysis device according to claim 12, wherein
if the second medically-related information acquired by the acquirer is unknown lesion information, the hardware processor classifies the second medically-related information into the first medically-related information, and
the hardware processor presents a classification result to a user for approval, and if the classification result is approved, the hardware processor learns the unknown lesion information as prescribed lesion information according to the classification result and registers the prescribed lesion information in the storage.

14. An analysis device comprising:
a hardware processor;
an acquirer; and
an outputter, wherein
the hardware processor acquires first medically-related information obtained through computer processing performed on medical information,
the acquirer acquires second medically-related information from structured data which is data based on the medical information that has been structured,
the hardware processor compares the first medically-related information acquired by the analyzer and the second medically-related information acquired by the acquirer, and
the outputter outputs next step information on the basis of a comparison result from the comparing by the comparison processor.

15. An analysis method comprising:
analyzing medical information through computer processing to acquire first medically-related information;
generating structured data by structuring unstructured data acquired from user-created information on the basis of the medical information;
acquiring second medically-related information from the structured data;
comparing the first medically-related information acquired by the analyzing and the acquired second medically-related information; and
outputting next step information on the basis of a comparison result from the comparing.

16. A recording medium storing a program causing a computer to perform:
analyzing medical information through computer processing to acquire first medically-related information;
generating structured data by structuring unstructured data acquired from user-created information on the basis of the medical information;
acquiring second medically-related information from the structured data;
comparing the first medically-related information acquired by the analyzing and the acquired second medically-related information; and
outputting next step information on the basis of a comparison result from the comparing by the comparison processor.
